(19) United States
(12) Patent Application Publication
(10) Pub. No.: US 2019/0018930 A1
(43) Pub. Date: Jan. 17, 2019

(54) METHOD FOR BUILDING A DATABASE

(71) Applicants: SYSMEX CORPORATION, Kobe-shi (JP); OSAKA UNIVERSITY, Osaka (JP)

(72) Inventors: Kazuki KISHI, Kobe-shi (JP); Kenichi SAWA, Kobe-shi (JP); Yasuto NAOI, Suita-shi (JP); Shinzaburou NOGUCHI, Suita-shi (JP)

(73) Assignees: SYSMEX CORPORATION, Kobe-shi (JP); OSAKA UNIVERSITY, Osaka (JP)

(21) Appl. No.: 15/884,462

(22) Filed: Jan. 31, 2018

(30) Foreign Application Priority Data
Jul. 12, 2017 (JP) 2017-136368

(51) Int. Cl. G06F 19/28; G06F 19/24; G16H 50/70; G06F 17/30

(52) U.S. Cl. CPC G06F 19/28; G06F 17/30312; G16H 50/70; G06F 19/24

(57) ABSTRACT

The present invention effectively utilizes data reflecting the expression of measurement target genes and non-measurement target genes other than the measurement target genes or functions of the gene products obtained by next generation sequencing analysis or microarray analysis.

A first embodiment of the invention for solving these problems is a method for constructing a database of gene related information including gene related measurement data reflecting expression of a gene in a biological sample or a function of a gene product, wherein the database is used for searching for a candidate for a new marker, the method comprising: a step of acquiring information specifying a gene to be analyzed; a step of acquiring information on a gene to be analyzed other than the gene to be analyzed A step of acquiring gene-related measurement data, a step of outputting gene-related information of the non-analysis target gene to a database, and a step of storing gene related information of the non-analysis target gene and biological sample information related to the biological sample which is information related to the biological sample from which the gene-related measurement data were acquired in the database.

### CROSS REFERENCE TO RELATED APPLICATIONS

This application claims priority from prior Japanese Patent Application Publication No. 2017-136368, filed on Jul. 12, 2017, entitled “METHOD FOR BUILDING A DATABASE”, the entire contents of which are incorporated herein by reference.

### FIELD OF THE INVENTION

The present invention relates to a method for building a database and a system for building a database.

### BACKGROUND

In recent years, attempts have been made to determine a treatment policy based on the molecular level of a patient, such as gene expression level, centering on breast cancer. For example, Japanese Patent Application Publication No. 2011-223957 describes a method for predicting the prognosis of breast cancer that is negative for lymph node metastasis and positive for estrogen receptor based on the expression of 95 genes.

The background for such prognostic predictions has been the rapid development of next generation sequencing and detection technologies and analytical techniques by microarrays and the like for comprehensively analyzing expression of genes across all genes.

### SUMMARY OF THE INVENTION

With next generation sequencing analysis and microarray analysis, it is now possible to analyze the expression levels of numerous genes and DNA sequence variations in DNA. NCBI Gene Expression Omnibus and other databases that can be used in the public domain are also being constructed. On the other hand, since the data accumulated in each database have not necessarily been collected under standardized conditions and analyzed, the database may contain analytical errors and the like, so that the state of gene expression and the like in the database is unlikely to genuinely reflect the gene expression of the samples. Further, neither the state of the individual collected samples nor the clinical contexts are homogeneous.

While the number of genes used to predict the prognosis of a disease and to predict the therapeutic effect of a drug is limited, in next-generation sequencing analysis and microarray analysis, genes and proteins that do not require measurement are also analyzed in large quantities.

In view of such problems in next-generation sequencing analysis and microarray analysis, the present invention provides a method to effectively utilize data reflecting the expression of measurement-target genes and non-target genes or functions of the gene products acquired by next generation sequencing analysis and microarray analysis.

A first embodiment of the invention for solving these problems is a method for constructing a database of gene related information including gene related measurement data reflecting expression of a gene in a biological sample or a function of a gene product, wherein the database is used for searching for a candidate for a new marker, the method comprising: a step of acquiring information specifying a gene to be analyzed; a step of acquiring information on a gene to be analyzed other than the gene to be analyzed A step of acquiring gene-related measurement data, a step of outputting gene-related information of the non-analysis target gene to a database, and a step of storing gene related information of the non-analysis target gene and biological sample information related to the biological sample which is information related to the biological sample from which the gene-related measurement data were acquired in the database.

A second embodiment of the invention for solving these problems is a method for searching for a candidate for a new marker based on gene related information including gene related measurement data reflecting the expression of the gene in the biological sample or the function of the gene product, wherein the method includes a step of acquiring information specifying an analysis target gene, a step of acquiring gene-related measurement data for a non-analysis target gene other than the analysis target gene, a step of outputting gene related information of the non-analysis target gene to a database, a step of storing in the database the gene related information of the non-analysis target gene and biological sample related information which is information related to the biological sample from which the gene related measurement data were obtained, a step of associating the gene related information with the biological sample related information, a step of acquiring, for each gene, a numerical value indicating the strength of relevance between the gene-related measurement data included in the gene-related information and the biological sample-related information, and a step of determining a candidate for a new marker as a gene strongly related to the biological sample related information based on the numerical value.

The 3-1th embodiment of the invention for solving these problems is a system 500 for constructing a database of gene related information including gene related measurement data reflecting the expression of a gene in a biological sample or the function of a gene product, wherein the database is used for searching candidates for a new marker, the system including an a laboratory facility information processing apparatus 20 and a laboratory facility database storage apparatus 100, wherein the laboratory facility information processing apparatus 20 acquires information specifying the analysis target gene, acquires the gene-related measurement data for a non-analysis target gene other than the analysis target gene, and stores the gene related information of the non-analysis target gene in the laboratory facility database storage apparatus, and the laboratory facility database storage apparatus 100 outputs gene related information of the non-analysis target gene and receives and stores biological sample-related information which is information related to the biological sample from which the gene-related measurement data was obtained.

The 3-2nd embodiment of the invention for solving these problems is a system 600 for constructing a database of gene related information including gene related measurement data reflecting the expression of a gene in a biological sample or the function of a gene product, wherein the database is used for searching candidates for a new marker, and the system includes a medical facility information processing apparatus 50, a laboratory facility information processing apparatus 20, a medical facility database storage apparatus 101, wherein the laboratory facility information processing apparatus acquires information for specifying an analysis target gene, acquires the gene-related measurement data for a non-analysis target gene other than the analysis target gene, and outputs the gene related Information of the non-analysis target gene to the medical facility database storage apparatus 101, and the medical facility information processing apparatus 50 outputs the biological sample related information which is information related to the biological sample from which the gene related measurement data were acquired to the medical facility database storage apparatus 101, and the medical facility database storage apparatus receives and stores the gene related information of the non-non-analysis target gene and biological sample related information.

The 3-3rd embodiment of the invention for solving the problem is a system 700 for constructing a database of gene-related information including gene-related measurement data reflecting the expression of a gene in a biological sample or the function of a gene product, wherein the database is used for searching candidates for new markers, and the system includes a medical facility information processing apparatus 50, a laboratory facility information processing apparatus 20, and a database storage apparatus 102, and the laboratory facility information processing apparatus 20 acquires the information for specifying the analysis target gene, acquires the gene-related measurement data for a non-analysis target gene other than the analysis target gene, and outputs the gene related information of the non-analysis target gene to the database storage apparatus, and the medical facility information processing apparatus 50 outputs the biological sample related information which is information related to the biological sample from which the gene related measurement data were acquired to the database storage apparatus, and the database storage apparatus 102 receives and stores the gene related information of the non-analysis target gene and the biological sample related information.

According to embodiments 1, 2, 3-1, 3-2, and 3-3, data reflecting the expression of the measurement target gene and a gene other than the measurement target gene or the function of a gene product obtained by next-generation sequencing analysis and microarray analysis can be effectively utilized.

A fourth embodiment of the invention for solving these problems is a method for constructing a database of gene related information including gene related measurement data reflecting expression of a gene in a biological sample or a function of a gene product, wherein the data stored in the database are used as training data or verification data of artificial intelligence for searching for a new marker, the method including a step of acquiring information specifying a measurement target gene, a step of acquiring gene related measurement data of the measurement target gene, a step of storing gene-related information of the measurement target in a database, and a step of storing information related to the biological sample from which the gene-related measurement data were acquired in the database. According to the present invention, a large amount of artificial intelligence training data or verification data can be provided.

A fifth embodiment of the invention for solving these problems is a method for constructing a database of gene related information including gene related measurement data reflecting expression of a gene in a biological sample or a function of a gene product, wherein the database is used for searching for a candidate for a new marker, the method including a step of acquiring gene-related information obtained for a plurality of genes including non-analysis target genes other than the analysis target gene from a laboratory facility information processing apparatus and/or a medical facility information processing apparatus, a step of acquiring biological sample related information which is information related to the biological sample from which the gene related measurement data were acquired from the laboratory facility information processing apparatus and/or the medical facility information processing apparatus, and a step of storing the gene related information and the biological sample related information in the database.

A sixth embodiment of the invention for solving these problems is a system 500, 600, 700 for constructing a database of gene related information including gene related measurement data reflecting expression of a gene in a biological sample or a function of a gene product, wherein the database is used for searching candidates for a new marker, the system including database storage apparatus 100, 101, 102, the database storage apparatus acquires gene-related information obtained for a plurality of genes including non-analysis target genes other than the analysis target gene from a laboratory facility information processing apparatus 20 and/or a medical facility information processing apparatus 50, and acquires biological sample related information, which is information related to the biological sample from which the gene-related information was obtained, from the laboratory facility information processing apparatus 20 and/or the medical facility information processing apparatus 50, and stores the gene-related information and the biological sample-related information. According to the fifth and sixth embodiments, data reflecting the expression of a measurement target gene and genes other than the measurement target gene, or the function of the gene product acquired by next-generation sequencing analysis or microarray analysis can be effectively utilized.

According to the invention, it is possible to effectively utilize data reflecting the expression of measurement target genes and genes other than the measurement target genes, or functions of the gene products acquired by next-generation sequencing analysis or microarray analysis.

### BRIEF DESCRIPTION OF THE DRAWINGS

FIG. 1 is a diagram showing an outline of a first embodiment of the present invention;

FIG. 2 is a diagram showing the flow from collection of a biological sample to pretreating of the sample for measurement;

FIG. 3 is a flowchart showing the process of constructing a database using pretreated products of a measurement sample;

FIG. 4 is a diagram showing a part of an analysis target gene to be analyzed of Curebest (registered trademark) 95GC Breast;

FIG. 5 is a diagram showing a gene to be analyzed other than the analysis target gene shown in FIG. 4 of Curebest (registered trademark) 95GC Breast;

FIG. 6 is a diagram showing an example of gene-related information;

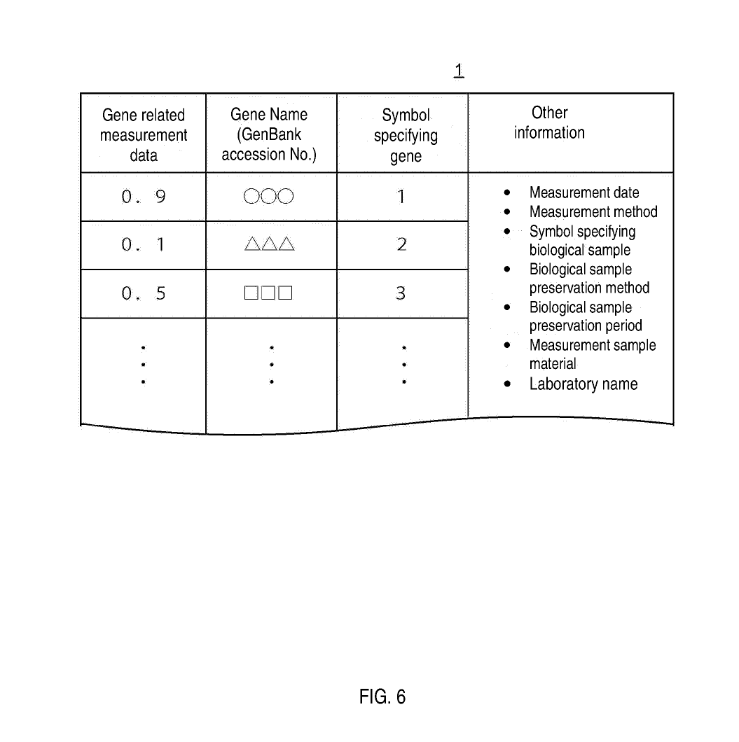

FIG. 7 is a diagram showing an example of biological sample related information;

FIG. 8 is a diagram showing an example of a report;

FIG. 9 is a flowchart showing the process of constructing a database of training data or verification data using a pretreated product of a measurement sample;

FIG. 10 is a diagram showing an outline of a database construction system according to 3-1th embodiment;

FIG. 11 is a diagram showing an overview of a database construction system according to 3-2nd embodiment;

FIG. 12 is a diagram showing an outline of a database construction system according to a 3-3rd embodiment;

FIG. 13 is a block diagram of a laboratory facility information processing apparatus;

FIG. 14 is a block diagram of a medical facility information processing apparatus;

FIG. 15 is a block diagram of first to third database storage apparatuses;

FIG. 16 is a flowchart showing a method of searching for a candidate for a new marker; and

FIG. 17 is a block diagram of a new marker candidate search apparatus.

### DESCRIPTION OF THE EMBODIMENTS OF THE INVENTION

Hereinafter, embodiments of the invention will be described in detail with reference to the accompanying drawings. Note that the method of constructing a database, the system for constructing a database, and the database storage apparatus according to the present invention are not limited to the specific embodiments described below. In the following description, the same reference numerals are assigned to the same components. m Therefore, descriptions of each component denoted by the same reference numeral can be shared between the same reference numerals. Furthermore, for terms commonly used in each embodiment, the explanation of terms in each embodiment are also applied to other embodiments.

1. Database Construction Method

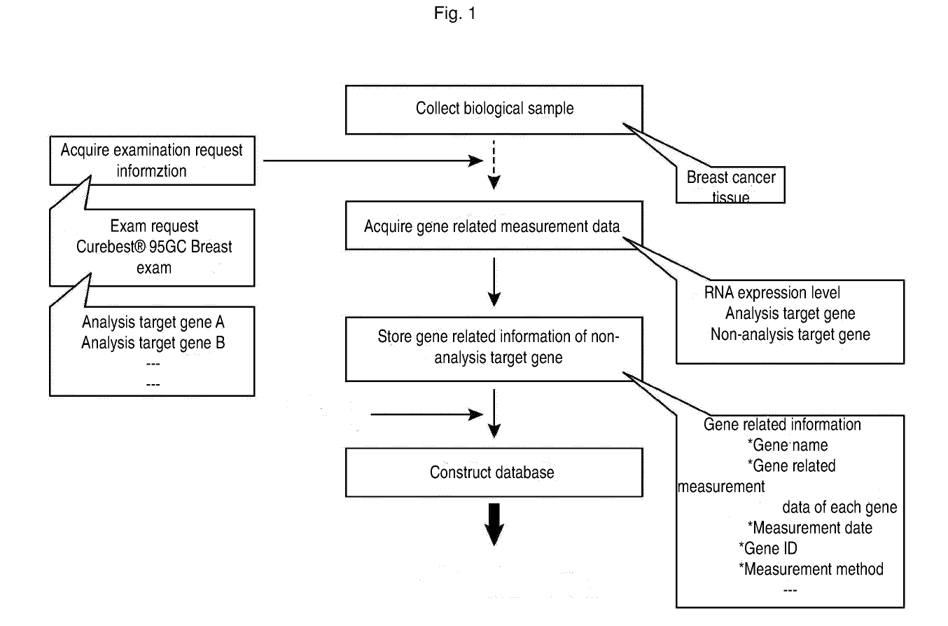

First, an outline of an embodiment of the present invention will be described with reference to FIG. 1. In an examination for determining the diagnosis of a disease, the prognosis of a disease, the necessity of medication or the like using the expression of a gene in a biological sample or the function of a gene product as an index, the embodiment constructs a database that stores gene related information 1 of non-analysis target genes other than the analysis target gene to be measured to achieve the objective of the examination. For example, when performing an examination with Curebest (registered trademark) 95GC Breast (Sysmex Corporation) using a breast cancer tissue as a biological sample, in general, gene-related measurement data are acquired such as the amount of expression of RNA and the like of the analysis target gene (95GC) contained in the examination item. In the present invention, the above-described gene-related measurement data are acquired for non-analysis target genes other than 95GC by the same method as that for measuring the amount of expression of RNA of 95GC, and gene-related information including gene-related measurement data of the non-analysis target gene are made into a database. These databases can be used, for example, for reanalysis (re-profiling) of the new marker in order to search for new markers such as disease biomarkers and therapeutic target molecules of diseases.

In addition, these databases can be used to provide training data and verification data for performing artificial intelligence machine learning when searching for the new marker or the like using artificial intelligence. The database also can be used to provide verification data for searching for new markers using statistical methods.

### [1-1. Construction of Database for Re-Profiling]

The first embodiment of the present invention relates to a method for constructing a database used for re-profiling for searching candidates for new markers. Specifically, the database nonvolatilely stores gene-related information including gene-related measurement data reflecting the expression of a gene or the function of a gene product in the biological sample.

The novel marker is, for example, a disease biomarker or a target molecule for the treatment of a disease. The disease biomarker can be used for disease risk assessment, screening, differential diagnosis, prognosis prediction, recurrence prediction and the like. The target molecule for the treatment of the disease also is a molecule that can prevent disease, treat disease, or delay disease progression by controlling the function of the target molecule. The target molecule also may be used to predict therapeutic effect.

### (1) Pretreating of Measurement Sample After Biological Sample Collection

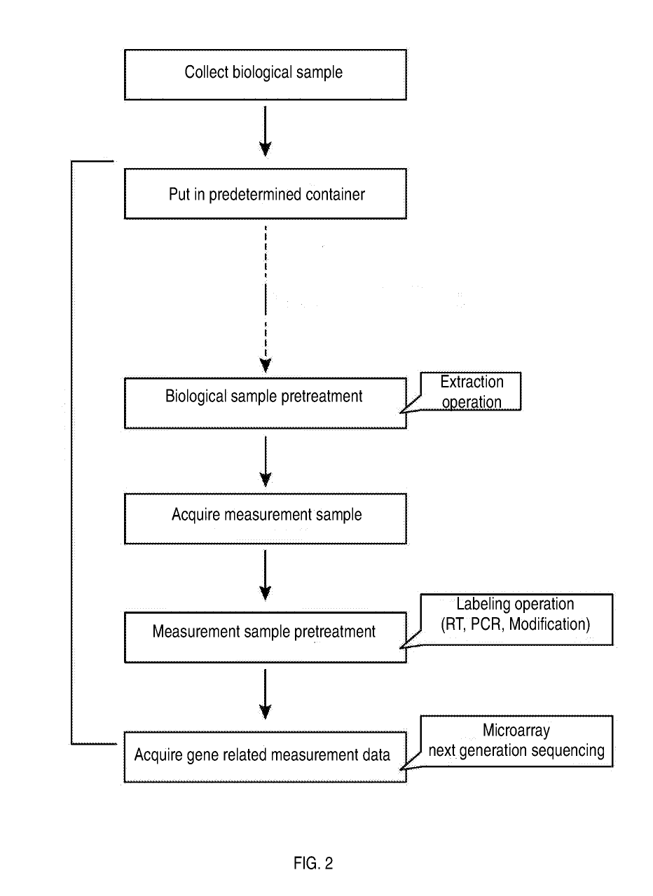

Next, referring to FIG. 2, the steps from the collection of the biological samples used to construct the database to the acquisition of the gene-related information will be described.

In the embodiment, the biological sample is not limited insofar as it is collected from a living body. For example, the biological sample may be a blood sample (whole blood, plasma, serum or the like), urine, body fluids (sweat, secretions from the skin, tears, saliva, spinal fluid, abdominal fluid, and pleural effusion), and tissues (fresh tissue, frozen tissue, fixed tissues, and tissues embedded in embedding agents such as paraffin).

It also is preferable that the biological sample is collected from at least one lesion selected from a group consisting of a predetermined disease, a predetermined disease type and a stage of a predetermined disease. The disease is not limited, but is preferably a tumor (a benign epithelial tumor, a benign non-epithelial tumor, a malignant epithelial tumor, a malignant non-epithelial tumor), more preferably a malignant epithelial tumor, or a malignant non-epithelial tumor, even more preferably malignant epithelial tumor, and yet more preferably a breast cancer. Most preferred is lymph node metastasis negative and estrogen receptor (ER) positive breast cancer.

The biological sample is preferably plural, and the plurality of biological samples are collected from lesions of different patients. More preferably, the plurality of biological samples are collected from lesions of the same disease in different patients, and still more preferably are collected from lesions of the same stage in different patients.

In a biological sample, a tissue considered to be normal which may serve as a negative control for the lesion site also may be collected. In this case, the tissue considered to be normal is preferably a normal part of the tissue to which the lesion site belongs. The normal part of the tissue to which the lesion site belongs may be taken from a plurality of patients or from a person not having the lesion.

The biological sample can be collected at the time of surgery or biopsy in a medical facility or the like to which the patient belongs. The collected biological sample is contained in a container such as a tube. A storage solution such as RNAlater (registered trademark) made by ThermoFisher Scientific Co., Ltd. or a fixative such as formaldehyde may be contained in the container. The biological sample contained in the container may be refrigerated or frozen. Although known preservatives or fixatives can be used for the preservation solution or the fixation solution, but from the viewpoint of preventing degradation and structural change of molecules in the biological sample during storage or transportation and keeping the biological sample in a certain state to some extent, it is preferable to use a commercially available kit or commercially available reagent. For example, a container attached to Curebest (registered trademark) 95GC Breast (Sysmex Corporation) can be used as a container for collecting a biological sample and a container for a biological sample. The biological sample contained in the container is pretreated in order to acquire gene-related measurement data at a medical facility or a laboratory facility that accepts an examination.

Examples of the gene-related measurement data reflecting the expression of the gene or the function of the gene product include the expression level of RNA (mRNA and/or microRNA) for each gene, the base sequence information of RNA, DNA (genomic DNA and/or mitochondrial DNA) methylation level, base sequence information of DNA (genomic DNA and/or mitochondrial DNA), or abundance of gene product protein (monomer protein, complex protein, monomeric peptide, and complex peptide), glycosylation modification information of proteins (including monomeric proteins, complex proteins, monomeric peptides, and complex peptides), and the like. For example, when the gene-related measurement data is the methylation amount of DNA, the gene-related measurement data includes at least the methylation amount of DNA in each gene and at least the position information of the methylation site of the DNA. When the gene-related measurement data is DNA sequence information, the gene-related measurement data also include not only base sequence information but also at least deletion, substitution, fusion, copy number mutation or the occurrence of insertion of the DNA base sequence of each gene, and information on the position thereof. The sequence information of the DNA also includes genetic polymorphism information such as single nucleotide polymorphism, double nucleotide polymorphism, triple nucleotide polymorphism and the like. When the gene-related measurement data is information on glycosylation modification of a protein, the gene-related measurement data also may include not only the presence or absence of modification of each protein but also the modification position of each protein, and information on the type of sugar chain of the modified protein are included.

Therefore, the pretreating of the biological sample from which the gene-related measurement data are acquired is not limited insofar as the RNA, DNA or protein of the measurement sample can be extracted in order to obtain the above-mentioned gene-related measurement data.

For example, when RNA is used to acquire gene-related measurement data, RNA can be obtained from a biological sample by a known method. Commercially available kits such as Qiagen RNeasy kit (registered trademark) manufactured by Qiagen can also be used for RNA extraction from a biological sample. When DNA is acquired to acquire gene-related measurement data, DNA also can be obtained from a biological sample by a known method. Commercially available kits such as QIAamp DNA Mini Kit (registered trademark) manufactured by Qiagen can also be used for DNA extraction from a biological sample. When proteins are used to obtain gene-related measurement data, proteins also can be extracted from biological samples by a known method. Commercially available reagents such as GE Healthcare Japan KK, trade name: Mammalian Protein Extraction Buffer and the like can be used for extracting proteins from biological samples. In the case where the biological sample is embedded in paraffin, it is possible to extract DNA from the biological sample using QIAamp DNA FFPE Tissue Kit (registered trademark) manufactured by QIAgen.

Regarding pretreating of biological samples, it is preferable to use commercially available kits or commercially available reagents from the viewpoint of preventing degradation of RNA and DNA in the process, structural change of proteins and the like, and homogenizing the sample for measurement.

Next, prior to acquiring the gene-related measurement data, the measurement sample may be pretreated as necessary. The pretreatment includes adding fluorescent labels, biotin labels or the like necessary for detection when acquiring gene-related measurement data to the RNA, DNA, or protein of the measurement sample, or the pretreatment product of the measurement sample described below. For example, when the measurement sample is RNA, the pretreatment of the measurement sample may include synthesizing cDNA or cRNA using RNA of the measurement sample as a template. Amplification of the cDNA or cRNA by PCR also may be included. In the case where the sample for measurement is DNA, the pretreatment of the sample for measurement may include amplifying the DNA of the sample for measurement by PCR if necessary. The pretreatment of the measurement sample also may include cutting the PCR product amplified using the DNA of the measurement sample or the DNA of the measurement sample as a template with a restriction enzyme. Where the sample for measurement is a protein, a surfactant such as sodium dodecyl sulfate, NP-40, Triton X-100, Tween-20 and/or a reducing agent such as β-mercaptoethanol, dithiothreitol or like reducing agent also may be included. The pretreatment methods are well known.

Also known is a method of labeling by fluorescence or biotin on the RNA, DNA, or protein of the measurement sample, or the pretreatment product of the measurement sample described below. For example, 3 'IVT PLUS Reagent Kit (trade name) manufactured by Thermo Fisher Scientific Co., Ltd. can be used.

The pretreatment product of the pretreated measurement sample according to the above method is subjected to measurement to acquire gene related measurement data.

It is desirable that the above-described collection of a biological sample, extraction of a sample for measurement from a biological sample, and pretreatment of a sample for measurement are carried out using a commercially available kit or commercially available reagents in unified form to manage quality in the various steps for the purpose of constructing a homogenized database.

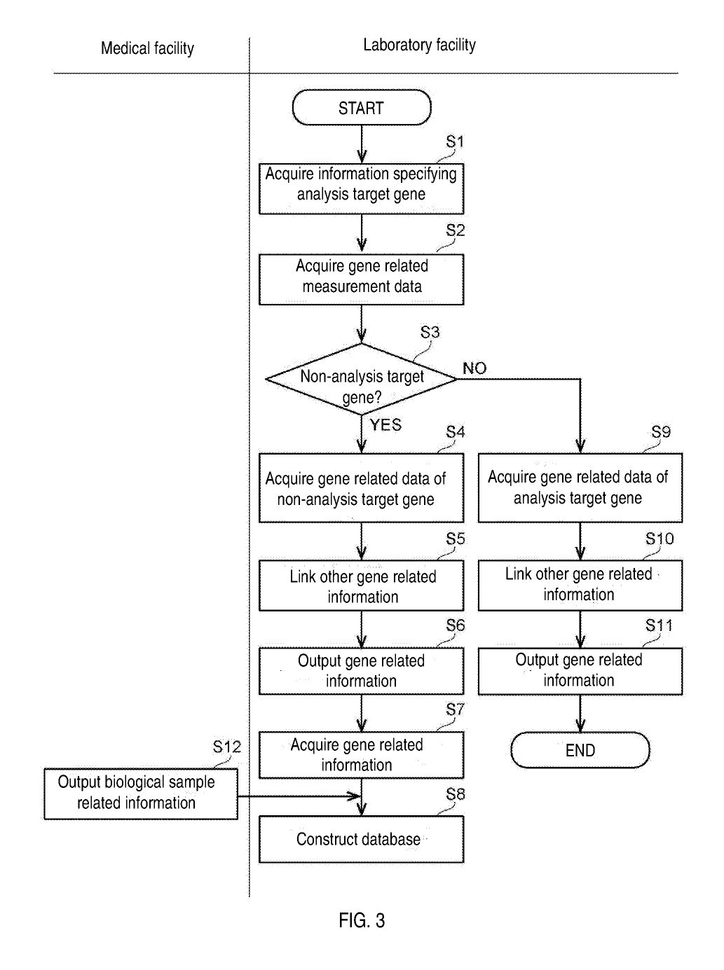

Next, each step for acquiring gene-related measurement data will be described with reference to FIG. 3. The acquisition of the gene-related measurement data may be performed by the laboratory facility information processing apparatus 20 according to the third embodiment which will be described later.

### (2) Acquisition of Gene-related Measurement Data

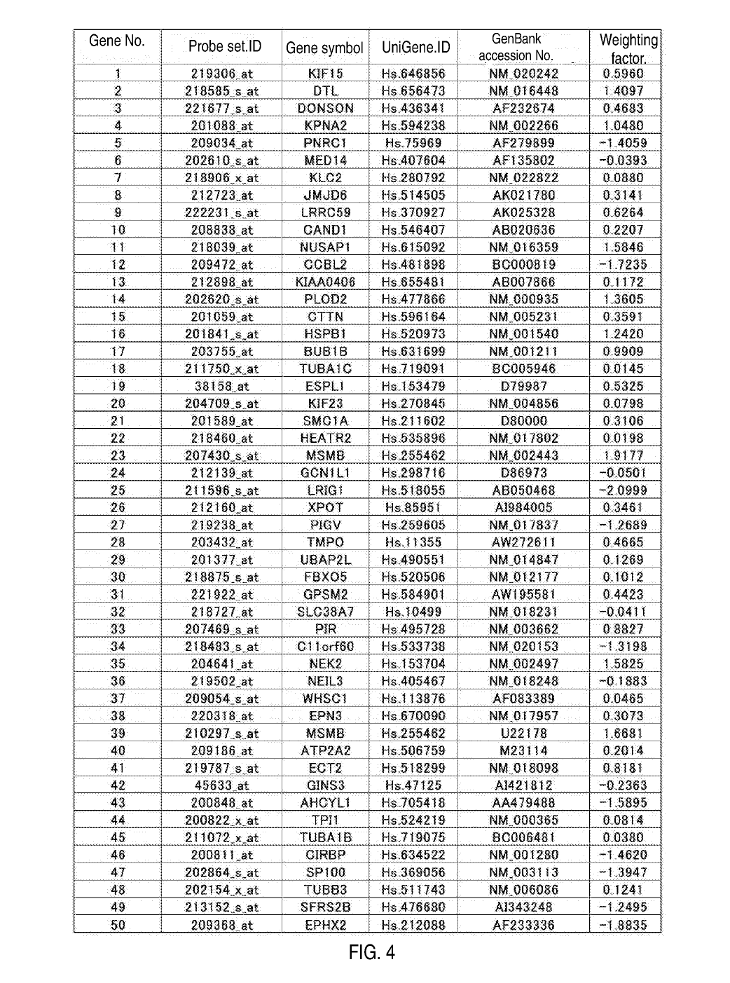

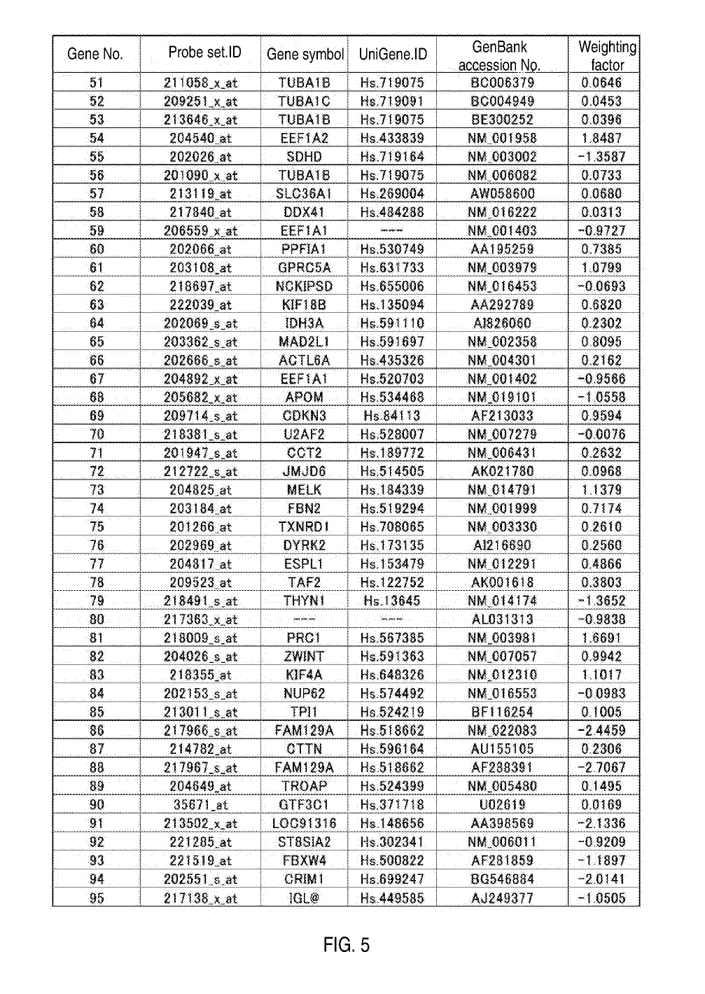

From the examination request form which the medical facility first fills in, the examiner or the processing section 21 of the laboratory facility information processing apparatus 20 (to be described later) acquires information for specifying the gene to be analyzed (step S1). For example, the analysis target gene may be one or a plurality of genes to be used for at least one analysis selected from a group consisting of disease risk determination, screening, differential diagnosis, prognosis prediction, recurrence prediction, efficacy prediction, and disease monitoring. It is preferable that the analysis target gene also is determined beforehand according to the analysis to be performed on each gene, for example, for each disease and for each disease stage in a laboratory and/or a medical facility. For example, taking Curebest (registered trademark) 95GC Breast as an example, a dedicated examination request form is attached to Curebest (registered trademark) 95GC Breast. The examination request form filled in with the required matter is sent by mail or on-line or the like from the medical facility to the laboratory facility. By receiving the inspection request form, the examiner of the laboratory facility grasps the Curebest (registered trademark) 95GC Breast as the inspection item and, if necessary, the processing unit 21 accepts the input information to start examination of the Curebest (registered trademark) 95GC Breast. Curebest (registered trademark) 95GC Breast is defined so that the 95 genes described in FIGS. 4 and 5 are to be analysis target genes. Therefore, the examiner or the processing unit 21 can specify that the analysis target genes of Curebest (registered trademark) 95GC Breast are the 95 genes described in FIGS. 4 and 5.

Here, the “probe set.ID” described in FIGS. 4 and 5 is a probe array in which, in a microarray (trade name: GeneChip (registered trademark) System) manufactured by Thermo Fisher Scientific Co., an ID number is attached to each of the probe sets including 11 to 20 probes fixed on a substrate. The base sequence of the nucleic acid (probe set) indicated by the probset.ID can be easily obtained from the web page https://www.affymetrix.com/analysis/netaffx/index.affx (database updated on Jun. 30, 2009). “UniGene.ID ” indicates the ID number of UniGene which is a database published by NCBI. The GenBank accession number indicates the accession number of a public database GenBank used for designing sequences of respective probes immobilized on a substrate in a microarray (trade name: GeneChip (registered trademark) System) manufactured by Thermo Fisher Scientific Co. The GenBank accession number indicates the number as of Jun. 30, 2009.

Next, in step S2, the examiner or the processing unit 21 acquires the gene-related measurement data by a predetermined measurement method. Methods for acquiring gene related measurement data are not limited. When the gene-related measurement data is the RNA expression level, RNA base sequence information, DNA methylation amount, or DNA base sequence information, it can be measured by base sequence sequencing and/or microarray. More specifically, in order to measure the expression level of RNA, RNA-seq analysis (Illumina, Inc.) using the next generation sequencer, and a microarray capable of RNA expression analysis, Human Genome U133 Plus 2.0 Array (by Thermo Fisher Scientific Inc.) and the like can be used. In order to measure the amount of DNA methylation, Infinium Methylation EPIC Kit (Illumina, Inc.) using microarrays or the like can be used. In addition, in order to measure (or detect) DNA sequence information, Genome-Wide Human SNP Array 6.0 or GeneChip (registered trademark) Human Genome U133 Plus 2.0 Array manufactured by Thermo Fisher Scientific Co., can be used for microarray measurement, exon sequence by next generation sequencer, and whole genome sequencing.

When the gene-related measurement data is the amount of protein present, it also can be measured by microarray and/or ELISA (including EIA). More specifically, it can be measured using an array of antibodies (C-series, G-series, L-series, Quantibody) and Protein Array series manufactured by RayBiotech.

Furthermore, when the gene-related measurement data is sugar chain modification of the protein, it can be measured by microarray and/or ELISA (including EIA). More specifically, it can be measured using a lectin array or the like manufactured by RayBiotech.

In step S2, if the sample for measurement or the product obtained by pretreating the sample is a nucleic acid, it may include thermal denaturation of these nucleic acids before performing the measurement.

From the viewpoint of maintaining the homogeneity of the acquired gene-related measurement data, it is preferable to select a measurement method in which the reproducibility of the gene-related measurement data is secured. For example, it is preferable to use a microarray and other measurement reagents consistently. In this way, by homogenizing the measuring method together with homogenization of the pretreated product of the measurement sample and/or measurement sample the quality of the gene-related measurement data can be kept constant. The laboratory that acquires the gene-related measurement data also is preferably a single facility (including a branch laboratory maintaining a certain examination accuracy) or one or more facilities to maintain consistent accuracy. The laboratory facility may be installed in a medical facility.

The acquisition of the gene-related measurement data by the above measuring method can be carried out by a measuring apparatus 10, which will be described later, suitable for measuring a signal such as fluorescence in each of the above measuring methods, the apparatus 10 acquires a signal in the above measurement and calculates the intensity of the light. The intensity of the signal also may be converted to amount of RNA (copy number), the amount of protein, the DNA methylation level or methylation percentage, the rate of change in the base sequence of RNA, the rate of change in the base sequence of DNA, the rate of protein glycosylation modification to acquire gene-related measurement data.

As shown in FIG. 4 or FIG. 5, the gene-related measurement data obtained by the above measuring method has at least a gene name (or GenBank accession number) or a code for identifying a gene (for example, GeneChip (registered trademark) System probeset.ID). Therefore, from the code for specifying the gene name or gene, the examiner or the processing unit 21 can identify which gene-related measurement data belongs to the non-analysis target gene (step S3), and the examiner or the processing unit 21 can acquire the gene-related measurement data of the non-analysis target gene (step S4).

Acquisition of the above-described gene-related measurement data may be performed only for non-analysis target genes other than the analysis target gene, but also may be performed for all analysis targets mounted on the microarray, total RNA, total DNA, or total protein may be measured; for example, only the gene-related measurement data of the non-analysis target gene may be extracted in the gene related measurement data. In step S5 of FIG. 3, in addition to the code for identifying the gene or gene name (or GenBank accession number), as well as the measurement date of the gene related measurement data, at least one selected from a group including of the measuring method, the amount of the measurement sample, the testing facility, the preservation method of the biological sample, the storage period of the biological sample, and at least one kind selected from a group including of other gene related information such as a code (for example, ID), are output to the first database storage apparatus 100, a second database storage apparatus 101, or a third database storage apparatus 102 (to be described later) by the examiner or the processing unit 21 (step S6).

It is preferable that the gene-related measurement data are acquired for a plurality of non-analysis target genes and/or a plurality of analysis target genes. The plurality of non-analysis target genes may be selected, for example, not only as genes to be analyzed but also genes suggested to be associated with a predetermined disease, a predetermined disease type, or a stage of a predetermined disease. The non-analysis target gene is a gene other than the analysis target gene and also is a gene which is analyzable in each of the above measuring methods.

According to the above method, the examiner or the processing unit 21 may also acquire the gene-related measurement data of the analysis target gene (step S9). Similarly to the gene-related measurement data of the non-analysis target gene, the gene-related measurement data of the analysis target gene is linked with other gene related information (step S10), and output to the first database storage apparatus 100, the second database storage apparatus 101, or the third database storage apparatus 102 (step S10).

The gene related data may be normalized or standardized and stored in the first database storage apparatus 100, the second database storage apparatus 101, or the third database storage apparatus 102. When the measurement method is a microarray, examples of normalization method include global normalization such as total intensity normalization, Lowess normalization, and/or local normalization. More specifically, the data can be normalized by the RMA algorithm, the MASS algorithm, the PLIER algorithm, or the like. As the analysis software using the RMA algorithm, the product Asymmetric Expression Console software (Thermo Fisher Scientific) may be mentioned. When the measurement method is a method using the next generation sequencer, Reads Per Million mapped reads (RPM), Read per kilobase of exon model per million mapped reads (RPKM), Trimmed mean of M values (TMM) method and the like may be mentioned.

The standardization of the above-mentioned gene-related data is carried out by comparing the data of housekeeping genes (GAPDH: glyceraldehyde-3-phosphate dehydrogenase, (β-actin, β-microglobulin, HPRT 1: hypoxanthine phosphoribosyltransferase 1 and the like.), or methods for comparing the values of gene-related measurement data based on expression levels of the gene product expression level, and performing statistical processing to determine a Z score, significance probability (p value), or likelihood using data recorded in the gene expression information database NCBI Gene Expression Omnibus (http://www.ncbi.nlm.nih.gov/geo/) of microarray experiments DataSet Record Data such as GDS 3834 (Multiple normal tissues) and the like as standardized values. It is also preferable that the data serving as the reference value is acquired by a homogenized method.

Examples of combinations of a plurality of genes to be analyzed include, for example, at least one selected from a group consisting of Curebest (registered trademark) 95GC Breast analysis target gene, Oncotype (registered trademark) DX analysis target gene, Mamma Print analysis target gene, Blue Print analysis target gene, PAM 50 analysis target gene, SureSelect Human All Exon V6 analysis target gene, SureSelect Human All Exon V6+COSMIC analysis target gene, SureSelect Human All Exon V6+UTR analysis target gene, SureSelect Human All Exon V5 target gene, SureSelect Human All Exon V5+UTRs target gene, SureSelect Human All Exon V5+IncRNA target gene, SureSelect Human All Exon V5+Regulatory target gene, TruSight Cancer target gene, TruSight Tumor 15 target gene, and TruSight Tumor 170 target gene.

Generally, the analysis target genes are about 20 genes to about 100 genes. However, the genes actually measured genes in microarrays and the like are about 38,500 genes, and analysis of 50,000 or more gene products including variants of gene products and the like is carried out. Therefore, when measuring the analysis target gene, the gene related information of the acquired non-analysis target gene and the biological sample related information corresponding thereto become extremely large. Therefore, the database that collects the information has a very large amount of information and is useful.

In acquiring the above-described gene-related measurement data, it is preferable to determine beforehand the type of examination criteria such as what type of biological sample is to be collected from a patient of any disease or stage, what kind of measurement method is used to acquire gene related measurement data, what collection site, how much sample to collect, how to collect the biological sample, how to preserve the biological sample until the measurement and the like, and acquire the gene related measurement data for a biological sample in conformance with these criteria. The examination criteria are selected from at least one type selected from a group consisting of the medical diagnosis related information, the medical treatment related information, the type of the biological sample, the measurement method, the amount of the biological sample to be measured, the biological sample collection method, and the biological sample storage method. The criteria may be determined by an laboratory facility and/or a medical facility.

(3) Construction of Database

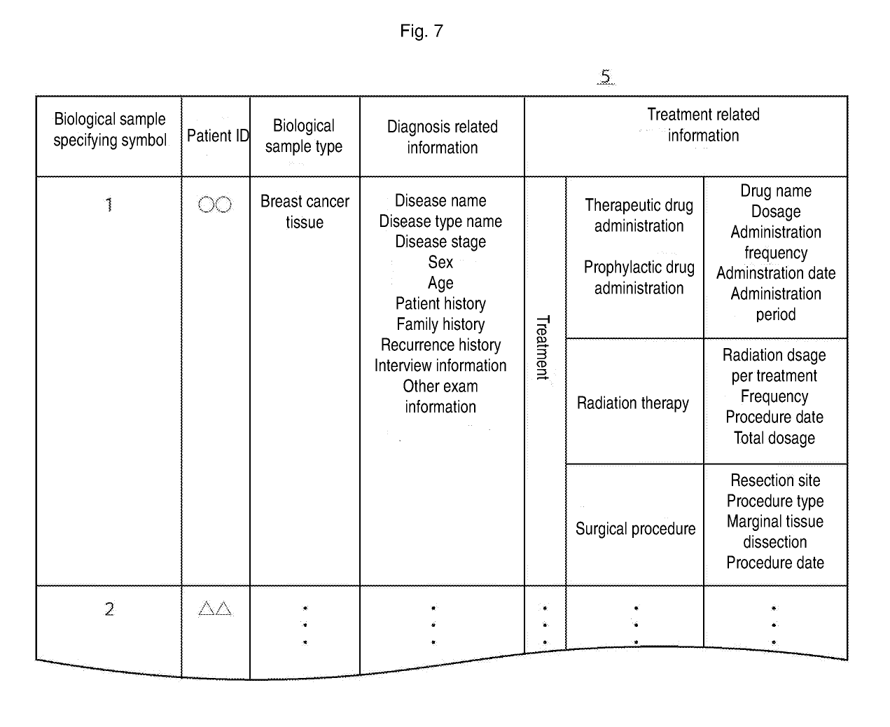

The processing unit 101 of the first database storage apparatus 100, the second database storage apparatus 101, or the third database storage apparatus 102 that stores the gene related information also acquires the gene related information output at step 6 of FIG. 3 (step S7), and stores the obtained gene-related information and the biological sample related information 5 obtained from the medical facility in step 12 in a nonvolatile manner (step S8). As shown in FIG. 7, the biological sample related information 5 includes at least a code for specifying a biological sample. The code (for example, ID) specifying a biological sample, may be a code (for example, a patient ID) for identifying a patient from which the biological sample is collected that is associated with a type of the biological sample. The biological sample related information 5 also includes at least one kind selected from a group consisting of diagnosis information related to the patient, and treatment related information. The medical diagnosis related information includes at least one of a disease name, a disease type name, a disease stage, a patient's sex, a patient's age, a patient's past history, a patient's family history, a recurrence history, a transition history, interview information, a menstrual history, and examination information other than gene related information. The treatment related information also includes at least one type of treatment history selected from a group consisting of, for example, administration of a therapeutic agent, administration of a prophylactic agent, radiation treatment ,and surgical treatment, as shown in FIG. 7. More specifically, when the treatment is administration of a therapeutic agent or administration of a prophylactic agent, the treatment history includes the name of the administered drug, the dose, the administration frequency, the administration date, the administration period and the like. When the treatment is radiotherapy, the treatment history includes the dose of radiation per dose, frequency, duration of treatment, total irradiation radiation dose and the like. When the treatment is a surgical treatment, the treatment history includes presence/absence of excision of surrounding tissues around the excision site such as the main excision site, surgical method, presence or absence of lymph node dissection of surrounding tissues such as lymph nodes, date of surgery and the like.

The gene related information and the biological sample related information 5 can correspond to each other using a code for specifying a biological sample as a key. Therefore, in the first database storage apparatus 100, the second database storage apparatus 101, or the third database storage apparatus 102, although the gene related information and the biological sample related information 5 are not necessarily combined in a single file, they may be combined in one file. As another aspect, the gene-related information and the biological sample related information 5 also may be individually stored in two database storage apparatuses that are accessible from a terminal of a user of a database, for example, via a network.

Furthermore, the database constructed in the present embodiment also may be stored in a storage medium such as an optical disk, or semiconductor memory element such as a hard disk, a flash memory, or an optical disk. The storage format of the database on the storage medium is not limited as long as the display device can read the database. Storage in the storage medium is preferably nonvolatile. In this case, the database construction method can be re-read as a manufacturing method of the storage medium storing the database.

(4) Other Embodiments

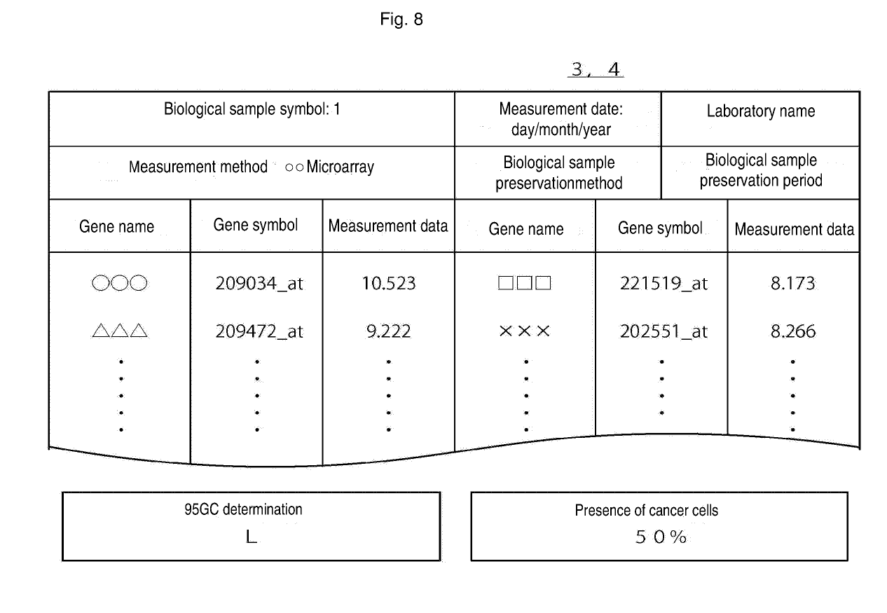

In the above database construction method, a step may be included in which reports 3 and 4 are prepared to report the gene related information 2 of the analysis target gene obtained in 1-1. (2) above, or the gene related information 2 of the analysis target gene and the gene related information 1 of the non-analysis target gene to a medical facility. The reports 3 and 4, for example, as shown in FIG. 8, include at least one type selected from a group consisting of a code for identifying the name of each gene (or GenBank accession number) and/or code for identifying each gene, gene related measurement data of each gene, a code for identifying a biological sample from which the gene-related measurement data was obtained, the measurement date of the gene-related measurement data, a measurement method, the name of the laboratory facility, the preservation method of the biological sample, and the storage period of the biological sample. The reports 3 and 4 also may contain at least one determination result selected from a group consisting of, for example, risk assessment of disease, screening, differential diagnosis, prognosis prediction, recurrence prediction, efficacy prediction, and disease monitoring. Curebest (registered trademark) 95GC Breast can predict the prognosis of breast cancer recurrence for susceptibility to preoperative chemotherapy of breast cancer, lymph node metastasis negative, and estrogen receptor (ER) positive breast cancer patients. From the prognosis prediction, it also is also possible to predict whether only hormonal therapy should be applied after surgery, or combined with chemotherapy. For example, in Curebest (registered trademark) 95GC Breast, report 3 shows that the prognostic result of breast cancer recurrence is H (relapse high-risk group) or L (relapse low-risk group) for Lymph node metastasis negative and estrogen receptor (ER) positive patients. In reports 3 and 4, a value indicating the content (presence or absence) of cancer cells for indicating whether the biological sample contained the amount of cancer cells necessary for examination may be displayed.

In the present embodiment, each step (step S1 to step S6, or step S1 to step S6, step S9 and step S10) performed by the processing unit 21 of the laboratory facility information processing apparatus 20 is executed by a computer program. Each step (steps S7, S12 and S8) performed by the processing unit 101 of the first database storage apparatus 100, the second database storage apparatus 101, or the third database storage apparatus 102 is also executed by a computer program. The computer program may be stored in a storage medium such as a hard disk, a semiconductor memory element such as a flash memory, or an optical disk. The storage format of the program in the storage medium is not limited insofar as the display apparatus can read the program. Storage in the storage medium is preferably nonvolatile.

In one example of the present embodiment, even if the biomarker of a disease searched by re-profiling is a biomarker of a disease different from the disease that the patient from whom the biological sample is taken, biomarker may be a biomarker of the same disease as the disease of the patient from whom the biological sample was taken.

According to the present embodiment, it is also possible to conduct the measurement under conditions that control the quality of measurement sample and gene related measurement data so as to homogenize the steps from collection of the measurement sample to the construction of the database. Since there is no need to consider quality defects of the measurement sample due to the preservation state of the biological sample, the gene-related measurement data acquired under the conditions of quality controlled in this manner reflect the state of the diseased tissue of the patient from whom the biological sample was collected. Thus, the database constructed according to the first embodiment is more reliable than other databases in that it reflects the condition of the patient's diseased tissue.

1-2. Construction of Database for Training Data and Verification Data]

According to a second aspect of the invention, a method is provided to construct a database to provide training data (also called teaching data, learning data) for classifying artificial intelligence into a discriminant, decision tree, nearest neighbor method, support vector machine, neural network, machine learning (also called teacher data, learning data) for machine learning such as deep learning, and a verification data (test data) for determining whether the constructed learning model is valid. The database constructed in the embodiment can be used for verification (validation) of a mathematical model obtained by statistical methods such as regression analysis, multiple regression analysis, variance analysis, principal component analysis and the like.

In the method for constructing a database of the invention as described in the first embodiment, it is possible to conduct the measurements under conditions that control the quality of the gene-related measurement data and measurement sample so as to homogenize the steps from collection of the measurement sample to the construction of the database. Therefore, the gene related measurement data of the analysis target genes and the non-analysis target genes acquired pursuant with the collection of a biological sample, pretreatment of the biological sample, the pretreatment method of a measurement sample obtained by such pretreatment, and the method of acquiring gene-related measurement data described in the first embodiment have higher reliability than that of other databases. Therefore, highly reliable data can be provided as verification data for determining whether training data or the constructed learning model is effective.

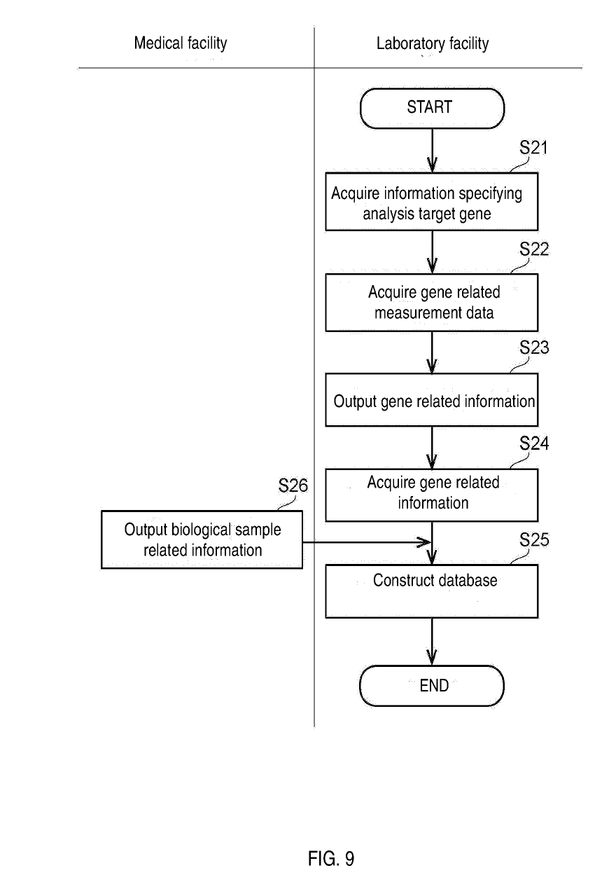

Specifically, the second embodiment as shown in FIG. 9 includes a step S21 in which the examiner or the processing unit 21 of the laboratory facility information processing unit 20 acquires information specifying the gene to be analyzed, step S22 in which the examiner or the processing unit 21 acquires the gene-related measurement data of the analysis target gene, and step S23 in which the gene related information 2 of the analysis target gene are output to the first database storage apparatus 100, the second database storage apparatus 101, or the third database storage apparatus 102. The second embodiment includes a step S26 in which the processing unit 101 of the first database storage apparatus 100, the second database storage apparatus 101, or the third database storage apparatus 102 acquires the gene related information output in step 23 (step S24), and stores the obtained gene-related information and the biological sample related information 5 acquired from the medical facility in step S25 in a nonvolatile manner.

Furthermore, the database constructed in the present embodiment also may be stored in a storage medium such as an optical disk, or semiconductor memory element such as a hard disk, a flash memory, or an optical disk. The storage format of the database on the storage medium is not limited as long as the display device can read the database. Storage in the storage medium is preferably nonvolatile. In this case, the database construction method can be re-read as a manufacturing method of the storage medium storing the database.

In the second embodiment, the examiner or the processing unit 21 may acquire the gene-related measurement data for the non-analysis target gene in step S22, and output the gene related information of the non-analysis target gene 1 to the first database storage apparatus 100, the second database storage apparatus 101, or the third database storage apparatus 102 in step S23, and store the gene related information 1 of the non-analysis target gene in the first database storage apparatus 100, second database storage apparatus 101, or the third database storage apparatus 102 in step S24. Also in the second embodiment, the database may be constructed from only the gene-related information 1 of the non-analysis target gene from step S22 to step S25.

In the present embodiment, each step (step S21 to step S23, or step S1 to step S23, step S26 and step S27) executed by the processing unit 21 of the laboratory facility information processing apparatus 20 is executed by a computer program by the processing unit of the first database storage apparatus 100, the second database storage apparatus 101, or each step (step S24, S26, and S25) is executed by the processing unit 101 of the third database storage apparatus 102 also by a computer program. The computer program may be stored in a storage medium such as a hard disk, a semiconductor memory element such as a flash memory, or an optical disk. The storage format of the program in the storage medium is not limited insofar as the display apparatus can read the program. Storage in the storage medium is preferably nonvolatile.

The database constructed by the above method can be used for artificial intelligence learning or to verify a model constructed by artificial intelligence. The gene related information 2 of the analysis target gene and the gene related information 1 of the non-analysis target gene stored in the database may be used to cause artificial intelligence to learn one or both depending on the purpose. For example, regarding one disease, gene related information 2 of an analysis target gene and biological material related information 5 corresponding thereto, which are stored in a database, also may be divided into two groups, one used as training data and the other used as verification data. The gene related information 2 of the analysis target gene used for Leave-One-Out Cross-Validation and the biological material related information 5 corresponding thereto can be handled as verification data even when performing Leave-One-Out Cross-Validation by using all the gene related information 2 of the analysis target gene stored in the database as training data for a single disease. In this section, the gene related information 2 of the analysis target gene can be replaced with the gene related information 1 of the non-analysis target gene.

2. System for Constructing Databases

The third embodiment of the present invention relates to a system for constructing the database described in the first embodiment and the second embodiment.

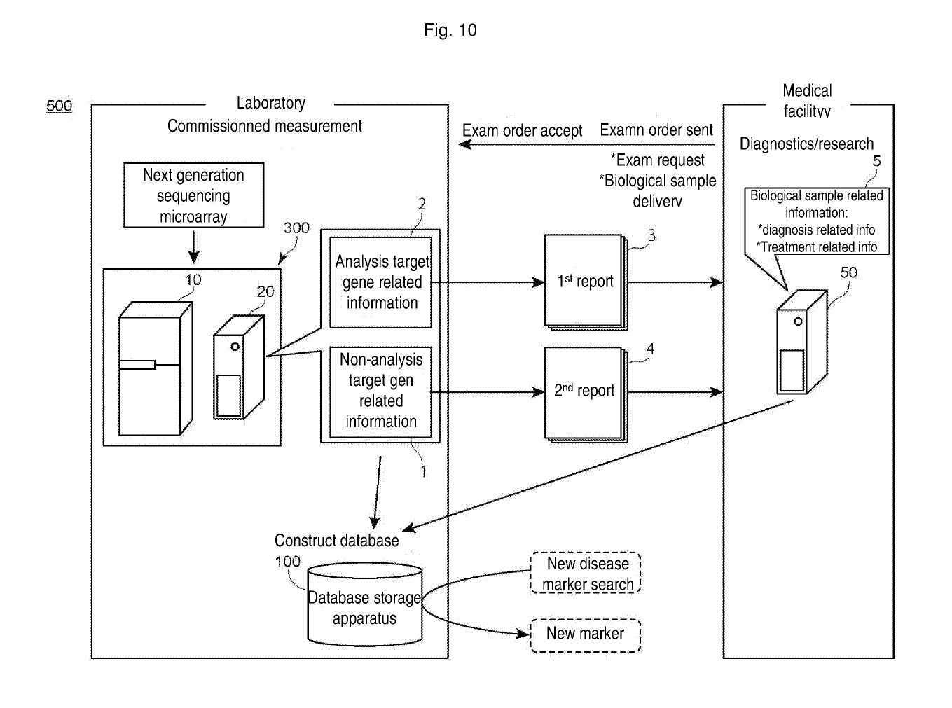

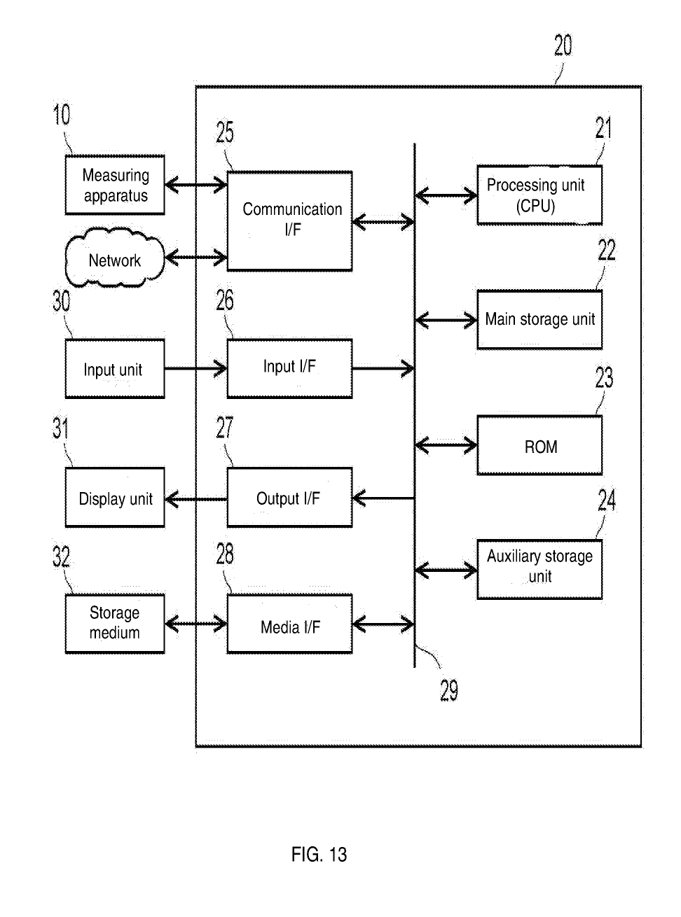

The embodiments of the third embodiment include the 3-1st embodiment for constructing a database in a laboratory, the 3-2nd embodiment for constructing a database in a medical facility, and the 3-3rd embodiment for constructing a database laboratory and the medical institution collaborate 3-3 embodiment in which a laboratory and medical facility collaborate for constructing the database. Below, a schematic view of the system shown in FIG. 10 to FIG. 12 and each embodiment will be described with reference to FIGS. 13 to 15.

2-1. Configuration of Hardware

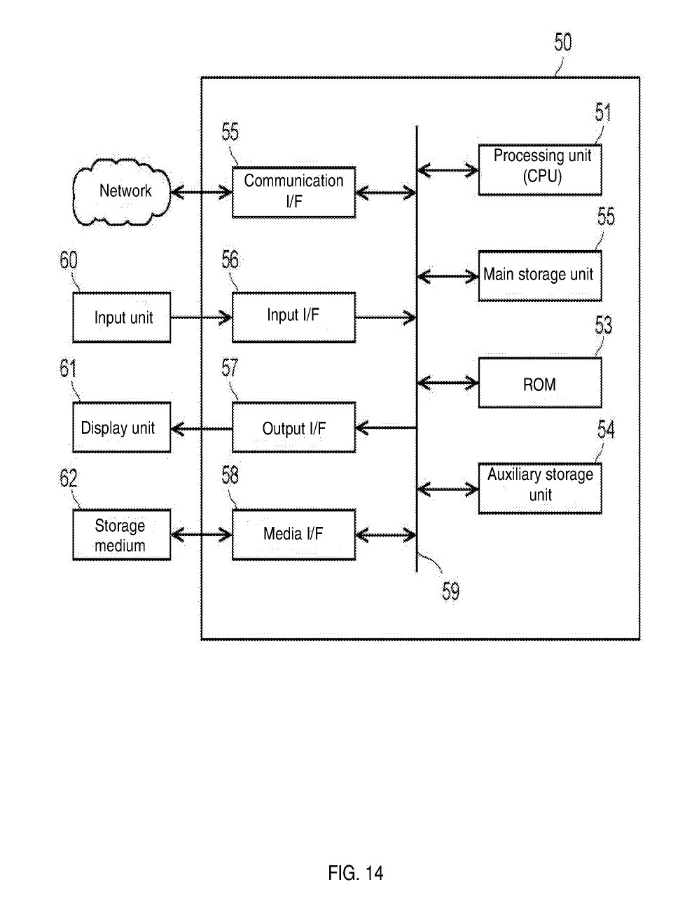

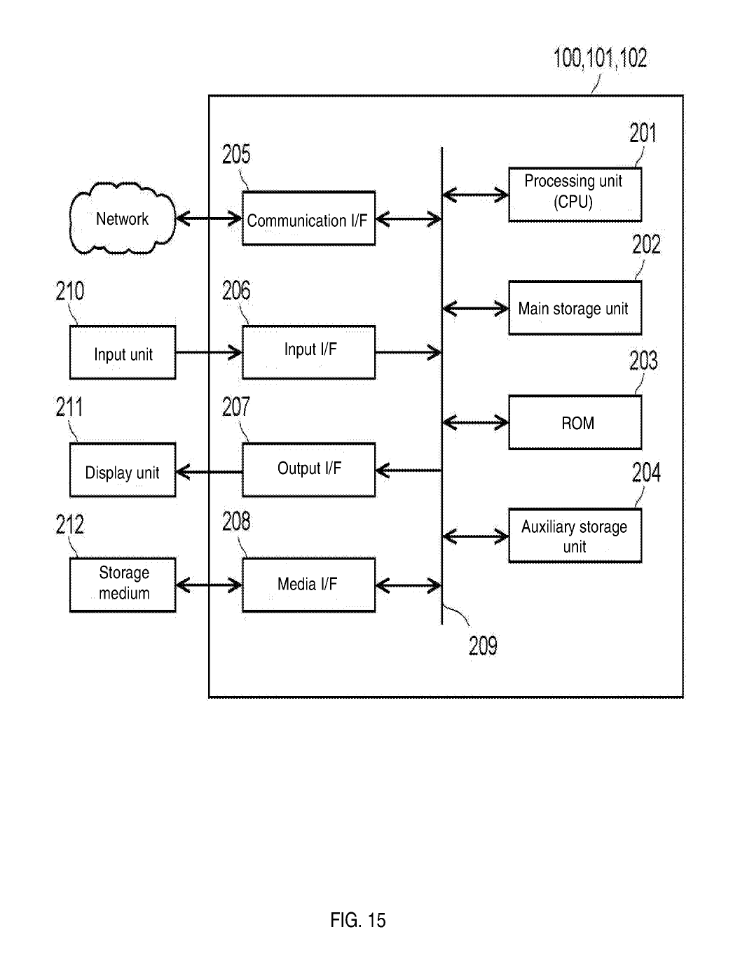

The laboratory facility information processing apparatus 20 shown in FIG. 13, the medical facility information processing apparatus 50 shown in FIG. 14, the first database storage apparatus 100, the second database storage apparatus 101, and the third database storage apparatus 102 shown in FIG. 15 are examples hardware structure. The hardware may be a personal computer, or a tablet type terminal. The hardware constituting the first database storage apparatus 100, the second database storage apparatus 101, and the third database storage apparatus 102 may have a role as a so-called server, and may be a CPU (Central Processing Unit) or an MPU (Micro-processing unit), which controls the storage apparatuses 100, 101, 102 using, for example, using a server operating system (OS) such as Linux (registered trademark), UNIX (registered trademark), Microsoft Windows Server.

The laboratory facility information processing apparatus 20 includes a processing unit (CPU) 21, a main storage unit 22, a ROM (read only memory) 23, an auxiliary storage unit 24, a communication interface (I/F) 25, an input I/F 26, an output I/F 27, a media I/F 28, a bus 29. The laboratory facility information processing apparatus 20 also includes an input unit 30 and a display unit 31. The laboratory facility information processing apparatus 20 also may include the storage medium 32.

The medical facility information processing apparatus 50 includes a processing unit (CPU) 51, a main storage unit 52, a ROM 53, an auxiliary storage unit 54, a communication I/F 55, an input I/F 56, an output I/F 57, a media I/F 58, a bus 59. The medical facility information processing apparatus 50 also includes an input unit 60 and a display unit 61. The medical facility information processing apparatus 50 also may include the storage medium 62.

The first database storage apparatus (laboratory facility database storage apparatus) 100, the second database storage apparatus (medical facility database storage apparatus) 101, and the third database storage apparatus 102 include a processing unit (CPU) 201, a main storage unit 202 a ROM 203, an auxiliary storage unit 204, a communication I/F 205, an input I/F 206, an output I/F 207, a media I/F 208, and a bus 209. The first database storage apparatus 100, the second database storage apparatus 101, and the third database storage apparatus 102 each have an input unit 210 and a display unit 211. The first database storage apparatus 100, the second database storage apparatus 101, and the third database storage apparatus 102 also may include the storage medium 212.

The CPUs 21, 51, and 201 control each unit based on the programs stored in the ROMs 23, 53, and 203 and the auxiliary storage units 24, 54, and 204. The CPUs 21, 51, and 201 also may be MPUs 21, 51, and 201.

The ROMs 23, 53, and 203 are configured by a mask ROM, a PROM, an EPROM, an EEPROM, and the like, and store programs and settings related to the hardware operation of the apparatuses and boot programs executed by the CPUs 21, 51, 201 during activation of the laboratory facility information processing apparatus 10, the medical facility information processing apparatus 50, the first database storage apparatus 100, the second database storage apparatus 101, and third database storage apparatus 102.

The main storage units 22, 52, and 202 are configured by a RAM such as SRAM or DRAM, and volatilely store information received from the input units 30, 60, and 210. The auxiliary storage units 24, 54, and 204 store application software and information input or generated during operation of the respective devices 20, 50, 100, 101, 102 in a nonvolatile manner (nonvolatile storage is also referred to as “recording”). The auxiliary storage units 24, 54, and 204 are configured by a semiconductor memory element such as a hard disk, a flash memory, an optical disk, or the like.

The communication I/Fs 25, 55, 205 receives information from an external device and also transmits information stored or generated by each device 20, 50, 100, 101, 102 to the outside. The communication I/Fs 25, 55, and 205 are serial interfaces such as USB, IEEE 1394, RS-232C and the like, parallel interfaces such as SCSI, IDE, IEEE 1284, analog interfaces including D/A converter, A/D converter, a network interface controller (NIC) and the like.

The input I/Fs 26, 56, and 206 accept character input, click input, voice input and the like from the input units 30, 60, and 210. For example, the input I/Fs 26, 56, and 206 are serial interfaces such as USB, IEEE 1394, and RS-232C, parallel interfaces such as SCSI, IDE, and IEEE 1284, and analog interfaces including a D/A converter and an A/D converter and the like. The accepted input content is stored in the main storage unit 22, 52, 202 or the auxiliary storage unit 24, 54, 204.

For example, the output I/Fs 27, 57, 207 are composed of the same interface as the input I/Fs 26, 56, 206, and output the information generated by the CPUs 21, 51, 201 to the display units 31, 51, 211. The output I/Fs 27, 57, 207 output the information generated by the CPUs 21, 51, 201 and stored in the auxiliary storage units 24, 54, 204 to the display units 31, 51, 211. Here, the display units 31, 51, and 211 may be a display or a projector, but may also be a printer.

The media I/Fs 28, 58, 208 read, for example, application software or the like stored in the storage media 32, 62, 212. The read application software and the like are stored in the main storage units 22, 52, 202 or the auxiliary storage units 24, 54, 204. The media I/Fs 28, 58, and 208 also write information generated by the CPUs 21, 51, and 201 to the storage media 32, 62, and 212. The media I/Fs 28, 58, and 208 write the information generated by the CPUs 21, 51, 201 and stored in the auxiliary storage units 24, 54, and 204 to the storage media 32, 62, 212. The storage media 32, 62, and 212 are configured by a flexible disk, a CD-ROM, a DVD-ROM, or the like. The storage media 32, 62, and 212 are connected to media I/Fs 28, 58, and 208 by a flexible disk drive, a CD-ROM drive, a DVD-ROM drive, or the like. The control of each hardware configuration by the CPU 21, 51, 201 is transmitted to each hardware configuration by buses 29, 59, 209.

2-2. System for Constructing a Database in a Laboratory Facility

As shown in FIG. 10, the system 500 according to the 3-1st embodiment includes a laboratory facility information processing apparatus 20 and a first database storage apparatus 100. The system 500 according to the present embodiment also may include the medical facility information processing apparatus 50. The laboratory facility information processing apparatus 20 may be connected to the measurement apparatus 10 directly or via a network to construct the measurement system 300. In the system, at least the laboratory facility information processing apparatus 20 and the first database storage apparatus 100 may be connected via a network. The laboratory facility information processing apparatus 20 and the medical facility information processing apparatus 50 also may be connected via a network.

The processing unit 21 of the laboratory facility information processing apparatus 20 acquires information specifying the analysis target gene, for example, by input from the input unit 30 or via the communication I/F 25, or the media I/F 28, and stores the information in the main storage unit 22, ROM 23, or the auxiliary storage unit 24. The processing unit 21 also acquires the gene-related measurement data from the measurement apparatus 10. Next, the processing unit 21 acquires gene related measurement data concerning the analysis target gene and/or the non-analysis target gene other than the analysis target gene, and generates gene related information for each gene. Subsequently, the processing unit 21 outputs the gene related information 2 of the analysis target gene and/or the gene related information 1 of the non-analysis target gene to the first database storage apparatus 100 via the communication I/F 25.

The processing unit 201 of the first database storage apparatus 100 acquires the gene related information 1 of the analysis target gene and/or the non-analysis target gene via the communication I/F 205. The processing unit 201 of the first database storage apparatus 100 also acquires biological sample related information 5, which is information related to the biological sample from which the gene related measurement data were acquired via input from the input unit 210 or through the communication I/F 205 or media I/F 208. The processing unit 201 of the first database storage apparatus 100 stores the acquired gene related information 2 of the analysis target gene and/or the gene related information 1 of the non-analysis target gene and the biological sample related information 5 in the auxiliary storage unit 204.

Here, the processing unit 21 of the laboratory facility information processing apparatus 20 also may store the information in the storage medium 32 in order to output the gene-related information 2 of the analysis target gene and/or the gene related information 1 of the non-analysis target gene to the first database storage apparatus 100. The processing unit 201 of the first database storage device 100 may acquire the gene related information 2 of the analysis target gene and/or the gene related information 1 of the non-analysis target gene via the media I/F 208. The processing unit 21 of the laboratory facility information processing apparatus 20 acquires the biological sample related information 5 and outputs the biological sample related information 5 together with the gene related information 2 of the analysis target gene and/or the gene related information 1 of the non-analysis target gene to the database storage apparatus 100. The description of each step of “1-1. Construction of database for re-profiling” is hereby incorporated by reference.

2-3. System for Constructing a Database in a Medical Facility

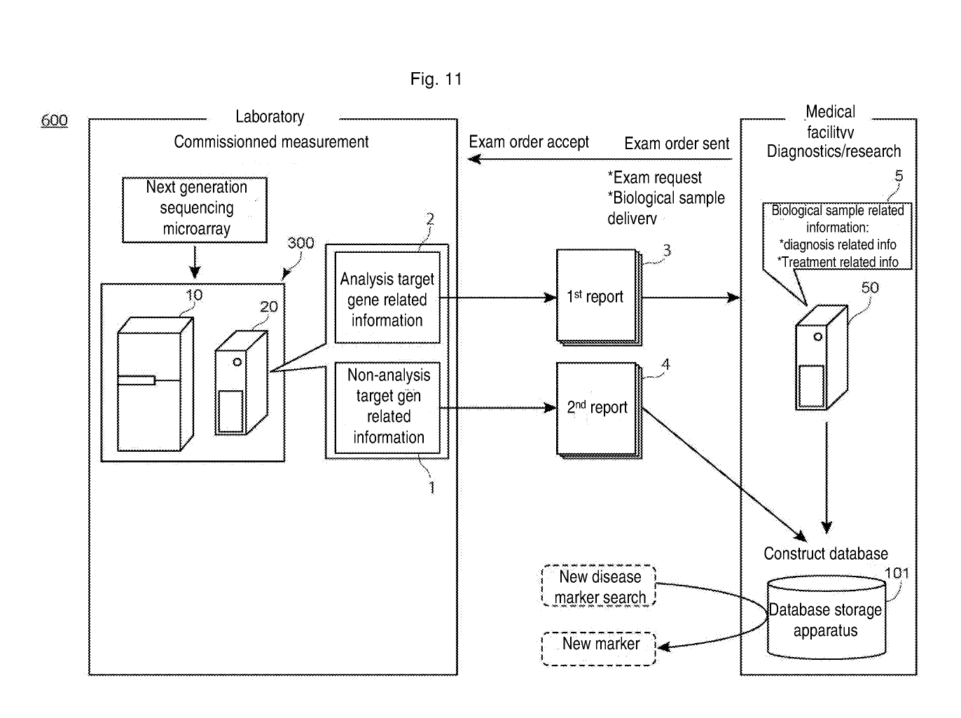

As shown in FIG. 11, the system 600 according to the 3-2nd embodiment includes a laboratory facility information processing apparatus 20, a medical facility information processing apparatus 50, and second database storage apparatus 101. In the system 600, the laboratory facility information processing apparatus 20, the medical facility information processing apparatus 50 and/or the second database storage apparatus 101 may be connected via a network.

The processing unit 21 of the laboratory facility information processing apparatus 20 acquires information specifying the analysis target gene, for example, by input from the input unit 30 or via the communication I/F 25, or the media I/F 28, and stores the information in the main storage unit 22, ROM 23, or the auxiliary storage unit 24. The processing unit 21 also acquires the gene-related measurement data from the measurement apparatus 10. Next, the processing unit 21 acquires the gene-related measurement data for non-analysis target genes other than the analysis target gene and/or the analysis target gene, and generates gene related information for each gene. Subsequently, the processing unit 21 outputs the gene related information 2 of the analysis target gene and/or the gene related information 1 of the non-analysis target gene to the second database storage apparatus 101 via the communication I/F 25.

The processing unit 51 of the medical facility information processing unit 50 receives the biological sample related information 5, which is information related to the biological sample from which the gene related measurement data, input from the input unit 60 by a doctor or the like in a medical facility, and outputs the biological sample related information 5 to the second database storage apparatus 101 via the communication I/F 55.

The processing unit 201 of the second database storage apparatus 101 acquires the gene related information 2 of the analysis target gene and/or the gene related information 1 of the non-analysis target gene via the communication I/F 205. The processing unit 201 of the second database storage apparatus 101 also acquires the biological sample related information 5 via the communication I/F 205 or the like. The processing unit 201 of the second database storage apparatus 101 stores the acquired gene related information 2 of the analysis target gene and/or the gene related information 1 of the non-analysis target gene and the biological sample related information 5 in the auxiliary storage unit 204.

Here, the processing unit 21 of the Laboratory facility information processing apparatus 20 stores the gene-related information 2 of the analysis target gene and/or the gene related information 1 of the non-analysis target gene in the storage medium 32 for output to the second database storage apparatus 101. The processing unit 51 of the medical facility information processing apparatus 50 also may store the biological sample related information 5 in the storage medium 52 in order to output the biological sample related information 5 to the second database storage apparatus 101. The processing unit 201 of the second database storage apparatus 101 acquires the gene related information 2 of the analysis target gene and/or the gene related information 1 of the non-analysis target gene and the biological sample related information 5 via the media I/F 208. The description of each step of “1-1. Construction of database for re-profiling” is hereby incorporated by reference.

2-4. System for Constructing Databases by Collaboration Between Laboratories and Medical Facilities

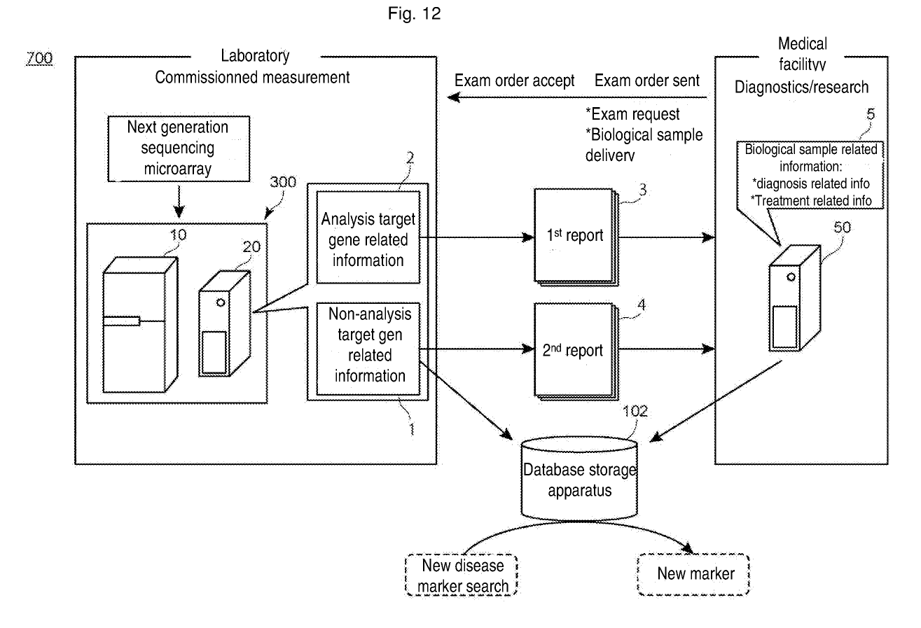

As shown in FIG. 12, the system 700 according to the 3-3rd embodiment includes a laboratory facility information processing apparatus 20, a medical facility information processing apparatus 50, and a third database storage apparatus 102. In the system 700, the laboratory facility information processing apparatus 20 and the third database storage apparatus 102, and/or the medical facility information processing device 50 and the third database storage apparatus 102 also may be connected via a network.

The processing unit 21 of the laboratory facility information processing apparatus 20 acquires information specifying the analysis target gene, for example, by input from the input unit 30 or via the communication I/F 25, or the media I/F 28, and stores the information in the main storage unit 22, ROM 23, or the auxiliary storage unit 24. The processing unit 21 also acquires the gene-related measurement data from the measurement apparatus 10. Next, the processing unit 21 acquires the gene-related measurement data for non-analysis target genes other than the analysis target gene and/or the analysis target gene, and generates gene related information for each gene. Subsequently, the processing unit 21 outputs the gene related information 2 of the analysis target gene and/or the gene related information 1 of the non-analysis target gene to the third database storage apparatus 102 via the communication I/F 25.

The processing unit 51 of the medical facility information processing unit 50 receives the biological sample related information 5, which is information related to the biological sample from which the gene related measurement data was obtained, input from the input unit 60 by a doctor or the like in a medical facility, and outputs the biological sample related information 5 to the third database storage apparatus 102 via the communication I/F 55.

The processing unit 201 of the third database storage apparatus 102 acquires the gene related information 2 of the analysis target gene and/or the gene related information 1 of the non-analysis target gene via the communication I/F 205. The processing unit 201 of the third database storage apparatus 102 acquires the biological sample related information 5 via the communication I/F 205 or the like. The processing unit 201 of the third database storage apparatus 102 stores the acquired gene related information 2 of the analysis target gene and/or the gene related information 1 of the non-analysis target gene and the biological sample related information 5 in the auxiliary storage unit 204.

Here, the processing unit 21 of the laboratory facility information processing apparatus 20 also may store the gene-related information 2 of the analysis target gene and/or the gene related information 1 of the non-analysis target gene in the storage medium 32 for output to the third database storage apparatus 102. The processing unit 51 of the medical facility information processing apparatus 50 also may store the biological sample related information 5 in the storage medium 52 in order to output the biological sample related information 5 to the third database storage apparatus 102. The processing unit 201 of the third database storage apparatus 102 acquires the gene related information 2 of the analysis target gene and/or the gene related information 1 of the non-analysis target gene and the biological sample related information 5 via the media I/F 208.

The description of each step of “1-1. Construction of database for re-profiling” is hereby incorporated by reference.

In the 3-1st embodiment, the 3-2nd embodiment, the 3-3rd embodiment, the processing unit 21 of the laboratory facility information processing apparatus 20 also may determine whether to generate reports 3 and 4 regarding the analysis target gene and/or non-analysis target gene.

3. Method for Searching for New Marker Candidate

The fourth embodiment of the invention relates to a method of searching for candidates of a new biomarker by reprofiling gene-related information including gene-related measurement data reflecting the expression of the gene in the biological sample or the function of the gene product using the database constructed according to the first embodiment. Therefore, the terms and description of the present embodiment common to the first embodiment are referred to the description of the first embodiment. The fourth embodiment also may be implemented by the new marker search apparatus 80 according to a fifth embodiment to be described later.

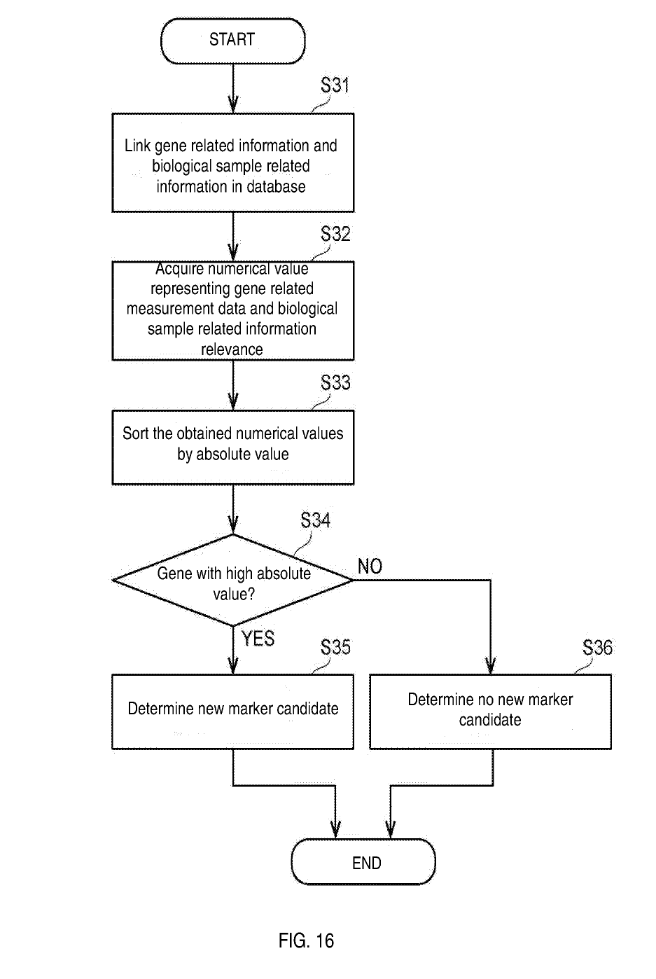

As shown in FIG. 16, in this embodiment the processing unit 81 of the examiner or the new marker searching apparatus 80 acquires gene related non-analysis target gene information 1 and biological sample related information 5 from the database storing the gene-related information 1 of the non-analysis target gene and the biological sample related information 5 in the first embodiment and, associates the gene related information 1 of the non-analysis target gene with the biological sample related information 5 for example, using information for identifying the biological sample included in both pieces of information as a key (step S31). Next, the examiner or processing unit 81 of the new marker search apparatus 80 acquires a numerical value indicating the strength of the relevance between the gene-related measurement data included in the gene related information and the biological sample related information 5 for each gene (step S32). For example, the numerical value may be determined based on the amount of RNA (copy number), the amount of protein, the level of DNA methylation or methylation, the rate of change of the base sequence of RNA, the rate of change of base sequence of DNA, the rate of glycosylation modification of protein. The numerical value also may be a statistically processed value such as RNA amount (copy number), protein amount, DNA methylation level or methylation rate, rate of change of RNA base sequence, rate of change of DNA base sequence, rate of glycosylation modification of protein, and the standardized data may be the numerical value. Specifically, the standardization is a significance probability (p value), a likelihood, a Z score, or the like. The statistical processing can be performed according to a known method. For example, the significance probability (p value) can be determined by significant difference test selected from Student's t test, Welch's t test, Wilcoxon's code rank test, and improved methods thereof. The likelihood can be obtained by a maximum likelihood estimation method, a likelihood test or the like. In the case of obtaining the Z score, The Z score can be determined according to Jung Kyoon Choi et al. (“Integration of Multiple Microarray Studies and Modeling of Inter-Study Validation (Combining multiple microarray studies and modeling interstudy variation “Bioinformatics, Volume 19, Supplement 1, 2003, p.i84-i90) using the package “GeneMeta v1.16.0” (http://www.bioconductor.org/packages/2.4/bioc/html/GeneMeta.html) included in the additional package collection “BioConductor” ver.2.4 used in the statistical analysis software “R”.

In statistical processing, for example, data such as DataSet Record GDS 3834 (Multiple normal tissues) or the like also can be used when reference data of a healthy tissue is required. When statistical analysis requires data as a criterion of disease, data registered in NCBI Gene Expression Omnibus (http://www.ncbi.nlm.nih.gov/geo/) also can be used. Preferably, reference data of healthy tissue or tissue from a disease lesion may be acquired according to the method of obtaining the gene-related measurement data in the first embodiment in order to obtain homogenized data.

Subsequently, the examiner or the processing unit 81 of the new marker searching apparatus 80 determines candidates for a new marker based on the numerical value with respect to each biological sample related information. Specifically, when the numerical value is an absolute value, the examiner or processing unit 81 of the new marker searching device 80, for example, sorts the gene-related measurement data corresponding to the absolute value on the basis of the absolute value (step S33), and determines which of the genes has a high absolute value (step S34). Then, the examiner or processor 81 of the new marker search unit 80 determines a gene having a high absolute value as a candidate for a new marker (step S35), and determines that the gene is a non-candidate for a new marker if the absolute value is low (step S36). The number of new markers may be plural.

In the case of obtaining the relevance between each biological sample related information and a plurality of genes, relevance can be obtained by subjecting the numerical values to statistical processing or the like. For example, multiple comparisons such as FALSE DISCOVERY RATE, Family-Wise error rate, Bonferroni method, Holm method and the like may be performed for a plurality of genes ranging from the highest in a predetermined ranking of the genes arranged based on the absolute values of the numerical values in step S33, and a performing method of estimating a gene having a relevance (a significant difference is recognized) of the biological sample related information by a resampling method such as Permutation test, Bootstrap method, Cross Validation or the like.

It is also possible to classify each gene for each biological function (for example, apoptosis-related genes and the like) and obtain the relationship between the function in the living body and each diagnosis related information or each treatment related information or the like. Such association can be obtained by Gene Set Enrichment Analysis or the like. Alternatively, after a group of genes strongly related to the biological sample related information is selected by hyper geometric distribution or the like, the relevance between each gene and biological sample related information can be obtained by using the degree of overlap of each gene group classified based on in vivo function as an index.

A candidate for a new marker also may be searched for based on the medical information related to, for example, the presence or absence of a family history, or the strength of the relation between the treatment related information such as whether the prognosis of the disease is good and the strength of the association of the gene. Such a search can be performed by statistical processing such as regression analysis, variance analysis, principal component analysis or the like using numerical values showing the relationship between the obtained gene-related measurement data and biological sample related information, or a hierarchical mathematical model may be obtained by cluster analysis such as clustering, k-means, mean-shift and the like, validated using a part of the obtained numerical value, and to determine from the validation data a plurality of genes having strong relevance from biological sample related information.

In the present embodiment, the processing unit 81 of the new marker search apparatus 80 performs each step (step S31 to S36) by executing a computer program. The computer program may be stored in a storage medium such as a hard disk, a semiconductor memory element such as a flash memory, or an optical disk. The storage format of the program in the storage medium is not limited insofar as the display apparatus can read the program. Storage in the storage medium is preferably nonvolatile.

4. New Marker Candidate Search Apparatus

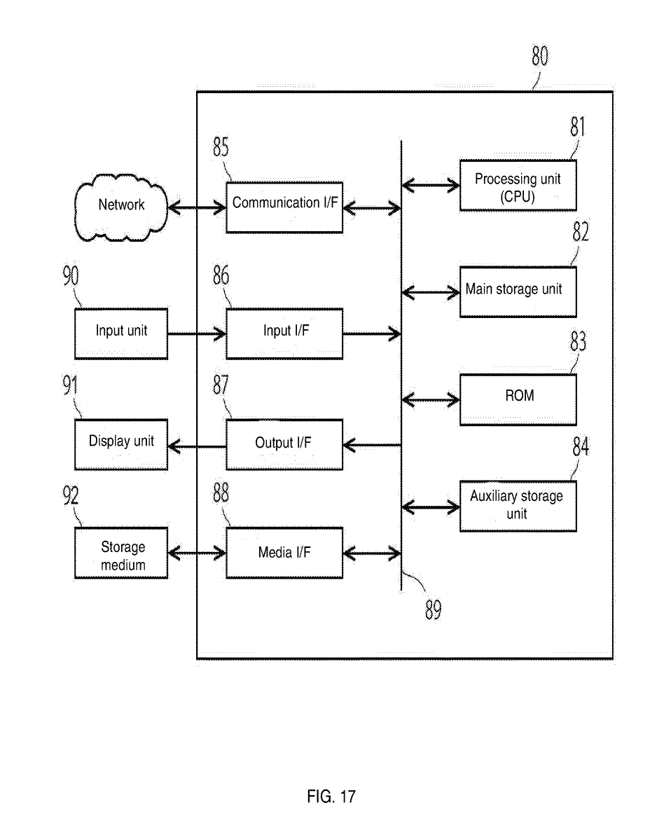

The new marker searching apparatus 80 shown in FIG. 17 is an example of a hardware configuration. The hardware may be a personal computer, or a tablet type terminal.

The new marker search apparatus 80 includes a processing unit (CPU) 81, a main storage unit 82, a ROM 83, an auxiliary storage unit 84, a communication I/F 85, an input I/F 86, an output I/F 87, and a media I/F 88. The new marker search apparatus 80 includes an input unit 90 and a display unit 91. The new marker search apparatus 80 also may include the storage medium 92. The description of each configuration incorporates the description of “2-1. Hardware Configuration” herein.

### EXPLANATION OF THE REFERENCE NUMERALS

20 laboratory facility information processing apparatus; 50 medical facility information processing apparatus; 100 first database storage apparatus; 101 second database storage apparatus; 102 third database storage apparatus; 500, 600, 700 system.

What is claimed is:

1. A method for constructing a database of gene related information including gene related measurement data reflecting expression of a gene in a biological sample or a function of a gene product, and
using the database to search for new marker candidates, the method comprising:
a step of acquiring information specifying an analysis target gene;
a step of acquiring the gene-related measurement data for a non-analysis target gene other than the analysis target gene;
a step of outputting gene related information of the non-analysis target gene to the database, and
a step of storing in the database the gene related information of the gene not to be analyzed and the biological sample related information which is information related to the biological sample from which the gene related measurement data was obtained.

2. The method according to claim 1, wherein
the gene to be analyzed is used for at least one analysis selected from a group including disease risk assessment, screening, differential diagnosis, prognosis prediction, recurrence prediction, efficacy prediction, and disease monitoring.

3. The method according to claim 1, wherein
the marker is a disease biomarker or a target molecule for the treatment of a disease.

4. The method according to claim 1, wherein
the biological sample is obtained from at least one type of foci selected from a group including a predetermined disease, a predetermined disease type and a stage of a predetermined disease.

5. The method according to claim 1, wherein
there are a plurality of biological samples, and the plurality of biological samples are collected from different diseased foci of different patients.

6. The method according to claim 1, wherein
the gene-related measurement data include at least one type selected from a group including RNA expression level, DNA methylation level, DNA base sequence information, RNA base sequence information, protein abundance amount, and protein glycosylation modification information.

7. The method according to claim 1, wherein
the gene-related measurement data are acquired by a predetermined measurement method.

8. The method according to claim 7, wherein
the predetermined measurement method is a base sequencing and/or a microarray measurement method when the gene-related measurement data are RNA expression level, DNA methylation level, DNA base sequence information, or RNA base sequence information;
the predetermined measurement method is microarray and/or ELISA when the gene-related measurement data are the protein abundance amount; and
the predetermined measurement method is microarray and/or ELISA when the gene-related measurement data are protein glycosylation modification.

9. The method according to claim 1, wherein
the gene-related measurement data are obtained in a single laboratory facility.

10. The method according to claim 1, wherein
the gene related information is at least one type selected from a group including the gene-related measurement data measurement date, the measurement method, the amount of the measurement sample, the examining facility, the preservation method of the biological sample, and the storage period of the biological sample, and the gene name of measurement data, a code for specifying GenBank accession number and/or gene, and a code for identifying a biological sample from which the gene-related measurement data are obtained.

11. The method according to claim 1, wherein
the biological sample related information includes at least one type selected from a group including medical care related information of the patient from whom the biological sample is collected, and treatment related information, and a code for specifying the biological sample, a code specifying the patient from whom the biological sample is collected, and the type of biological sample.

12. The method according to claim 1, comprising
a step of obtaining the gene-related measurement data for the analysis target gene.

13. The method of claim 12 further comprising:
a step of outputting gene related information of the non-analysis target genes to the database; and
a step of storing gene related information of the analysis target gene in a database.

14. The method according to claim 1, comprising:
a step of preparing a report of gene related information of the analysis target gene.

15. The method of claim 14, wherein the report comprises:
at least one determination result selected from a group including risk determination of disease, screening, differential diagnosis, prognosis prediction, recurrence prediction, efficacy prediction, and disease monitoring;
a code for specifying each gene name and/or each gene;
gene-related measurement data for each gene;
a code for specifying a biological sample from which the gene-related measurement data are acquired; and
at least one type selected from a group including the date of measurement, the method of measurement, the laboratory facility, the method of preservation of the biological sample, and the period of storage of the biological sample are included in the gene-related measurement data.

16. The method according to claim 1, wherein
there are a plurality of non-analysis target genes.

17. The method according to claim 2, wherein
the disease biomarker is a biomarker of a disease different from a disease afflicting the patient from whom the biological sample was taken.

18. The method according to claim 2, wherein
the disease biomarker is a biomarker of the same disease as a disease afflicting the patient from whom the biological sample was taken.

19. A method for searching a candidate for a new marker on the basis of gene-related information including gene-related measurement data reflecting expression of a gene in a biological sample or a function of a gene product, the method comprising:
a step of acquiring information specifying an analysis target gene;
a step of acquiring the gene-related measurement data for a non-analysis target gene other than the analysis target gene;
a step of outputting gene related information of the non-analysis target gene to the database;
a step of storing in the database the gene related information of the non-analysis target gene and the biological sample related information which is information related to the biological sample from which the gene related measurement data was acquired;
a step of associating the gene related information with the biological sample related information;
a step of acquiring, for each gene, a numerical value indicating the strength of the relevance between the gene related measurement data included in the gene related information and the biological sample related information; and
a step of determining that a gene strongly related to the biological sample related information based on the numerical value as a candidate for a new marker.

20. A method for constructing a database of gene related information including gene related measurement data reflecting expression of a gene in a biological sample or a function of a gene product, and
wherein the data stored in the database are used as artificial intelligence training data or verification data for searching for new markers, the method comprising:
a step of acquiring information specifying an analysis target gene;
a step of acquiring gene-related measurement data for the analysis target gene;
a step of storing gene-related information of the analysis target gene in the database; and
a step of storing biological sample related information, which is information related to the biological sample from which the gene related measurement data are acquired, in the database.

21. A method for constructing a database of gene related information including gene related measurement data reflecting expression of a gene in a biological sample or a function of a gene product, and
using the database to search for new marker candidates, the method comprising:
a step of acquiring the gene related information acquired for a plurality of genes including non-analysis target genes other than the analysis target gene from the laboratory facility information processing apparatus and/or the medical facility information processing apparatus;
a step of acquiring biological sample related information, which is information related to the biological sample from which the gene related measurement data was acquired, from the laboratory facility information processing apparatus and/or the medical facility information processing apparatus; and
a step of storing the gene related information, and the biological sample related information.
